(19) United States
(12) Patent Application Publication
(10) Pub. No.: US 2004/0209891 A1
(43) Pub. Date: Oct. 21, 2004

(54) Treatment of type 2 diabetes with inhibitors of dipeptidyl peptidase IV

(76) Inventors: Pierre Broqua, Southhampton (GB); Beatrice Sudre, Geneva (CH); Michael L Aubert, Geneva (CH)

Correspondence Address: FOLEY AND LARDNER, SUITE 500, 3000 K STREET NW, WASHINGTON, DC, 20007, US

(21) Appl. No.: 10/474,676

(22) PCT Filed: Apr. 10, 2002
(86) PCT No.: PCT/GB02/01674

(30) Foreign Application Priority Data
Apr. 11, 2001 (GB) 0109146.1

(51) Int. Cl. A61K 31/501; A61K 31/506; A61K 31/401

(52) U.S. Cl. 514/252.05; 514/256; 514/343; 514/423

(57) ABSTRACT

A method of treating an individual at risk of developing type 2 diabetes, or in the early stages thereof, so as to delay the onset and progression of the disease, which comprises the administration to the individual of repeated doses of an inhibitor of dipeptidyl peptidase IV or a prodrug thereof.

### FIELD OF THE INVENTION

The present invention relates to a method for delaying the onset of type 2 diabetes and alleviating the physiological consequences of type 2 diabetes.

### BACKGROUND

Diabetes mellitus affects about 5% of the population, and type 2 diabetes, also known as non-insulin dependent diabetes mellitus, accounts for more than 80% of all cases. Type 2 diabetes is particularly prevalent in obese people aged over 40. Complications of type 2 diabetes include retinopathy and nephropathy, and diabetics have a significantly increased chance of suffering cardiovascular disease.

A number of drugs are available for the treatment of type 2 diabetes, but new ones, particularly those acting by novel mechanisms, are still needed. One such class of candidate therapeutic agents comprises inhibitors of dipeptidyl peptidase IV (DP-IV, EC.3.4.14.5). These compounds act, at least in part, by blocking the inactivation of endogenous incretins such as GLP-1 and GIP, resulting in an increased sensitivity to insulin and reduced post-prandial hyperglycaemia. To date, however, these compounds have only been examined as a method for controlling the management of blood glucose levels on an acute basis. The implications of long-term treatment with these compounds have not been considered.

### SUMMARY OF THE INVENTION

We have now found that chronic treatment with inhibitors of DP-IV in a standard animal model of type 2 diabetes results in a delay in the development of the disease. Accordingly, one aspect of the present invention is a method of treating individuals at risk of developing type 2 diabetes, or in the early stages thereof, so as to prevent the progression of the disease, which method is to administer to the said individual repeated doses of a pharmaceutical composition comprising an inhibitor of DP-IV. Another aspect of the invention is a pharmaceutical composition for use in such treatment. A third aspect of the present invention is the use of inhibitors of DP-IV to prepare such compositions.

### DETAILED DESCRIPTION OF THE INVENTION

We have examined the effects of chronic treatment of Zucker Diabetic Fatty (ZDF) rats with inhibitors of DP-IV. The ZDF rat is a well known model for human type 2 diabetes. ZDF rats are hyperphagic, and when fed on a high fat diet they become diabetic, as shown by hyperglycaemia, hypertrigyceridaemia, polydipsia and an increase in circulating free fatty acids. Disease onset is observed at about 8 weeks and the animals are fully diabetic by 11 weeks of age. We found that chronic treatment of ZDF rats with inhibitors of DP-IV leads to a significant delay in the onset of the diabetic state, which indicates that such chronic treatment will be useful in human subjects at risk of developing type 2 diabetes, or in the early stages of the disease.

Accordingly, a first aspect of the present invention is a method of treating an individual at risk of developing type 2 diabetes, or in the early stages thereof, so as to delay the onset and progression of the disease. The treatment comprises the administration to the said individual of repeated doses of an inhibitor of DP-IV.

The assessment that an individual is at risk of developing type 2 diabetes will generally be made by an experienced physician, who will take into consideration such factors as the age and weight (and more specifically the body mass index, BMI) of the individual, as well as any history of diabetes in the individual's family and other risk factors. Similarly, a diagnosis of early-stage type 2 diabetes will be made by an experienced physician on the basis of a number of standard analyses and tests.

The inhibitor of DP-IV may be any compound that inhibits the enzymatic activity of DP-IV at a pharmacologically relevant dose. Suitable compounds can be identified as those that significantly inhibit the enzymatic activity of DP-IV in an in vitro assay at concentrations below 10 μM. Particularly suitable compounds are those that inhibit the enzymatic activity of DP-IV at concentrations below 0.1 μM. Such activity can be easily determined by one skilled in the art using one of the published assays. Suitable compounds should in addition preferably be selective, i.e. they should not significantly inhibit other unrelated enzymes at a concentration equal to that at which they inhibit DP-IV, and more preferably they should not inhibit such enzymes at a concentration ten-fold greater, even more preferably one hundred-fold greater, than that at which they significantly inhibit DP-IV.

The scope of the present invention extends to the use of prodrugs of DP-IV inhibitors. Prodrugs are well known in the art. A prodrug is a compound that is generally inactive per se, but which is subject to chemical or metabolic modification after administration, which modification causes the release of the active pharmaceutical agent. Prodrugs are typically used to increase oral bioavailability or to prolong the duration of action of a compound.

Examples of suitable compounds and methods for their preparation are disclosed in, for example, International Patent Applications WO91/16339, WO93/08259, WO95/15309, WO98/19998, WO99/46272, WO99/61431, WO99/67278, WO99/67279 and WO01/14318; U.S. Pat. Nos. 5,462,928, 5,543,396, 5,939,560, 6,011,155, 6,107,317, 6,110,949, 6,124,305, 6,166,063 and 6,201,132; and European patent applications 0 528 858, 0 610 317, 0 731 789, 1 043 328, 1 050 540 and 1 082 314.

In a preferred embodiment of the present invention, the inhibitor of DP-IV is an α-aminoacyl pyrrolidide, an α-aminoacyl thiazolidide, an α-aminoacyl pyrrolidinenitrile, or an α-aminoacyl thiazolidinenitrile. In a more preferred embodiment, the inhibitor of DP-IV is a compound according to general formula 1 or general formula 2, or a pharmaceutically acceptable salt of either of these.

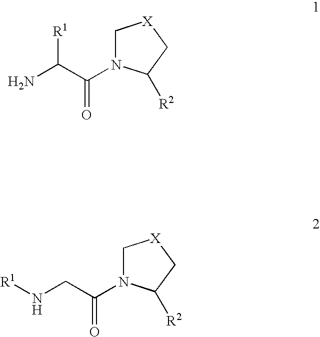

In these general formulae, X is selected from a methylene group CH₂ and a sulphur atom S; R¹ is selected from C₁-C₆ alkyl groups, including branched and cyclic alkyl groups, and (CH₂)ₙR³; R² is selected from a hydrogen atom H and a nitrile group CN; R³ is selected from NH-Het and NHCO-Het; Het is a pyridyl, pyrimidyl or pyrazinyl group that is optionally substituted with up to two groups independently selected from methyl, Cl, F, CN and CF₃; and n is 2, 3, 4 or 5.

The compounds according to general formulae 1 and 2 all have at least one basic nitrogen atom and so are able to form addition salts with protic acids. Examples of such acids include hydrochloric acid, sulphuric acid, phosphoric acid, acetic acid, trifluoroacetic acid, fumaric acid, maleic acid, citric acid, benzoic acid, pamoic acid and methanesulphonic acid. Insofar as these acids are pharmaceutically acceptable, such salts are included within the scope of the present invention.

The compounds according to general formula 1 have a stereogenic centre (asymmetric carbon atom) in the aminoacyl group. When R² is a nitrile, the compounds according to both general formulae have a stereogenic centre in the five-membered ring. Accordingly, these compounds can exist as optical isomers such as enantiomers and diastereomers. All such isomers are included within the scope of the present invention. The preferred stereochemistry is that illustrated in general formulae 3 and 4.

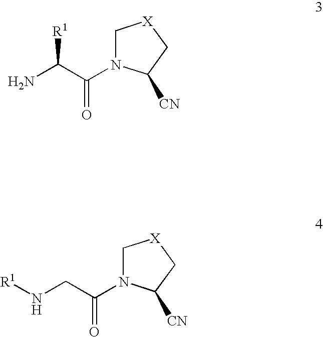

In a preferred embodiment of the present invention, the inhibitor is a compound according to general formula 1. More preferably, it is a compound according to general formula 1 wherein R¹ is a C₄ branched alkyl group such as sec-butyl or tert-butyl. Most preferably it is such a compound wherein X is CH₂ and R² is a nitrile, or X is S and R² is H.

In another preferred embodiment of the present invention, the inhibitor is a compound according to general formula 2. More preferably, it is a compound according to general formula 2 wherein R¹ is (CH₂)ₙR³, n is 2 and R³ is NH-Het. Most preferably it is such a compound wherein X is CH₂, R² is a nitrile, and Het is 5-cyano-2-pyridyl.

In the method of treatment according to the present invention, the inhibitor of DP-IV will be administered to the individual as a pharmaceutical composition such as, for example, a tablet, capsule, powder, suppository, solution or suspension. The general principles for the preparation of such formulations are well known in the art. The formulation may further comprise such pharmaceutically acceptable excipients as bulking agents, binding agents, preservatives, solvents, flavoring agents and the like. It may further include one or more additional pharmacologically active agents, such as anti-diabetic agents, but preferably the DP-IV inhibitor is the sole active agent.

The formulation may be administered by any appropriate route, including oral, buccal, sublingual, rectal, intravaginal and transdermal administration as well as by intravenous, subcutaneous and intramuscular injection. Preferably the formulation is administered orally as a tablet or capsule.

The dose will be determined by the attending physician, taking into consideration all the relevant factors. Typically a single dose will comprise between 1 mg and 1000 mg, preferably between 5 mg and 250 mg. The dose may be given once per day or more often, such as twice or three times per day. Treatment will be continued for an extended period of time such as several weeks, months or even years.

Alternatively, the formulation may be administered as a depot which releases active compound over a period of between one week and three months. Such controlled-release formulations are known in the art, and generally comprise a pharmaceutically active species associated with a biocompatible polymer. The polymer may simply encapsulate the active agent, forming a physical barrier to its release into the general circulation, or there may be a chemical association, such as a covalent or ionic interaction, between the polymer and the active agent. Such formulations are generally administered by intramuscular or subcutaneous injection. In this case, the administration will be repeated at intervals of one week up to three months so as to maintain treatment over an extended period.

A second aspect of the present invention is a pharmaceutical composition for the treatment of a person at risk of developing type 2 diabetes, or in the early stages thereof, so as to delay or prevent the progression of the disease. The composition comprises an inhibitor of DP-IV as described above, suitably formulated, together with instructions for repeated dosing.

A third aspect of the present invention is the use of an inhibitor of DP-IV for the preparation of a pharmaceutical composition for the treatment of a person at risk of developing type 2 diabetes, or in the early stages thereof, so as to delay or prevent the progression of the disease.

### EXAMPLES

### Example 1

### Preparation of Inhibitors of DP-IV

Inhibitors of DP-IV can be prepared according to published methods.

### Example 1A (2S)-1-((2′S)-2′-amino-3′,3′-dimethylbutanoyl)pyrrolidine-2-carbonitrile hydrochloride

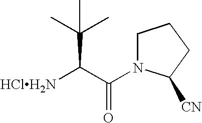

The title compound is prepared according to the methods of WO95/15309, and particularly of Example 13 therein. Briefly, BOC-protected tert-butylglycine is coupled to prolineamide, the primary amide function is dehydrated with trifluoroacetic anhydride to give the nitrile and the BOC-group is removed with HCl in dioxan.

### Example 1B

### (2S)-1-((2′S)-2′-amino-5′-pyrazinecarbonylaminopentanoyl)pyrrolidine-2-carbonitrile trifluoroacetate

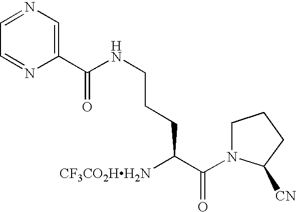

The title compound is prepared according to the method of Example 1A above. Briefly, N^{α}-BOC-protected N^{ω}-pyrazinecabonylornithine is coupled to prolineamide, the primary amide function is dehydrated with trifluoroacetic anhydride to give the nitrile, and the BOC-group is removed with trifluoroacetic acid.

### Example 1C

### N-Isoleucylthiazolidine Hydrochloride

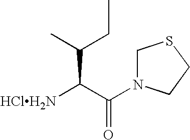

The title compound is prepared according to the standard methods. Briefly, BOC-protected isoleucine is coupled to thiazolidine and the BOC-group is removed with HCl in dioxan.

### Example 1D

### (2S)-1-((2′-(5″-Cyano-2″-pyridylamino)ethylamino)acetyl)pyrrolidine-2-carbonitrile

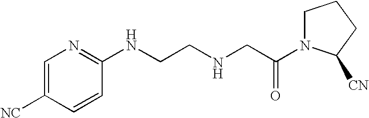

The title compound is prepared according to the methods of WO98/19998, and particularly of Example 3 therein. Briefly, bromoacetyl bromide is reacted with prolineamide and the product is dehydrated with trifluoroacetic anhydride to give N-bromoacetylpyrrolidine-2-carbonitrile. This is treated with 2-(5-cyano-2-pyridylamino)ethylamine to give the product.

### Example 2

### Inhibition of DP-IV In Vitro

The in vitro inhibitory action of the compounds is determined in a fluorimetric assay. Human DP-IV is incubated with a standard substrate, Ala-Pro-AFC, in the presence of various concentrations of the inhibitor. The reaction is monitored by measuring the increase in fluorescence due to the reaction product, AFC. Using standard manipulations, an inhibitory constant, Kᵢ, is determined. Typical results are given below.

| Compound of Example No. | Kᵢ (nM) |
|---|---|
| 1A | 1.0 |
| 1B | 0.4 |
| 1C | 33.0 |
| 1D | 5.0 |

### Example 3

### Effect of Long-Term Inhibition of DP-IV in ZDF Rat

Male ZDF rats, aged 6.5 weeks at the beginning of the study (day 0), are given the compound of Example 1A (10 mg/kg p.o.) once or twice per day for four weeks. Control animals are given vehicle. A group of untreated lean rats is used as a comparison. Glycaemia, insulinaemia, body weight, food and water intake, and plasma triglyceride and free fatty acid levels are monitored throughout the study.

### Example 3A

### Glycaemia

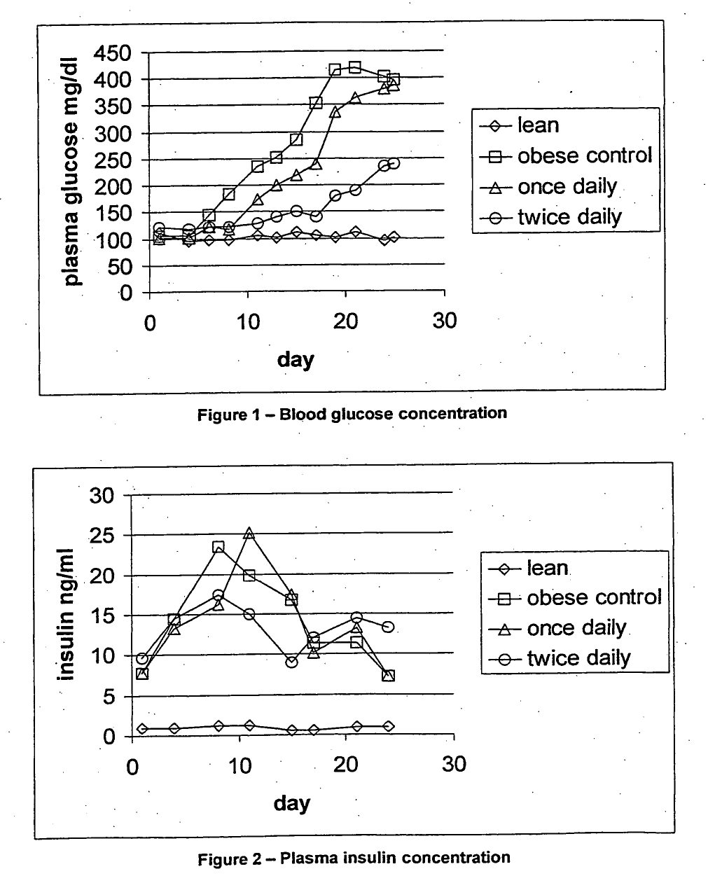

At the start of the study glycaemia is not significantly different in the obese animals compared to the lean rats. At day 8, the obese control group develop hyperglycaemia, which increases and reaches a plateau by day 19. The animals treated once daily with the inhibitor do not develop significant hyperglycaemia until day 15, and those treated twice daily do not develop significant hyperglycaemia until day 24. The results are presented in FIG. 1.

### Example 3B

### Insulinaemia

All three groups of obese animals show elevated plasma insulin concentrations at the beginning of the study period. In the control obese animal group, the insulin concentration rises rapidly to reach a peak by day 8 before decreasing as the islet β-cells die. In the group treated once daily with the inhibitor a similar pattern is observed, but the peak insulin level is only reached on day 11. In the group treated twice daily with the inhibitor insulin concentration does not attain the same high level, and there is evidence of β-cell survival at the end of the study period. The results are presented in FIG. 2.

### Example 3C

### Body Weight Gain

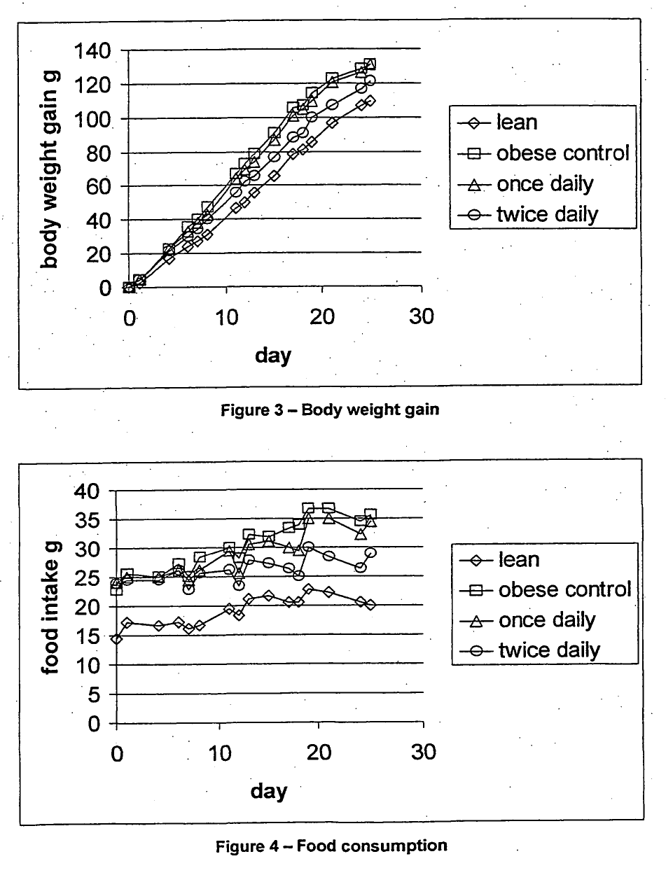

All three groups of obese animals gain weight faster than the lean group, but the group treated twice daily with the inhibitor gain less weight than the control obese group and the group treated once daily. The results are presented in FIG. 3.

### Example 3D

### Food and Water Intake

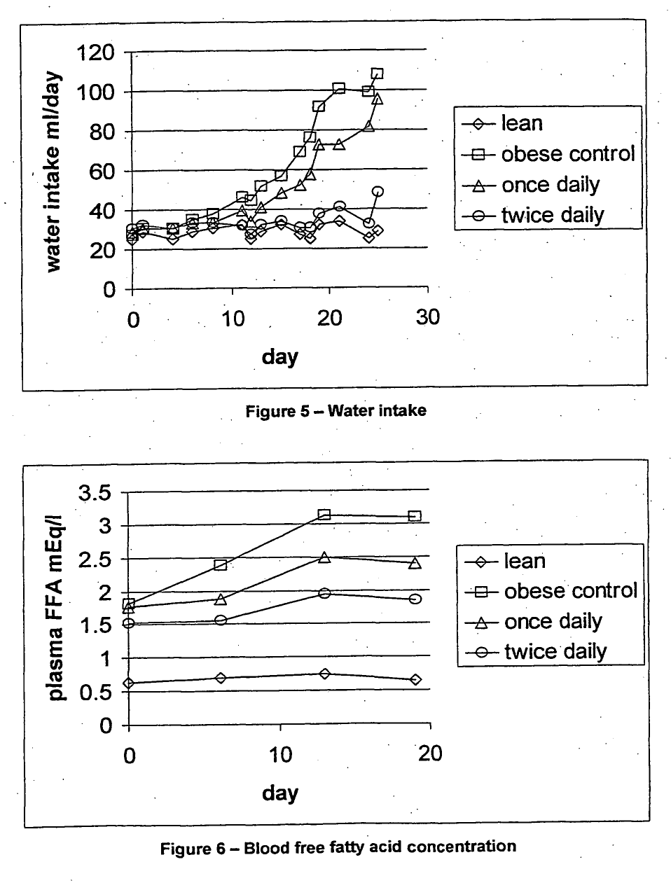

All three groups of obese animals eat more than the lean group, but from day 17 the food intake for the group treated twice daily is significantly less than that for the control obese group and the once-daily treatment group. From day 10, the control and once-daily treatment groups show an increase in their water consumption, but the twice-daily treatment group maintains a normal water intake. The results are presented in FIGS. 4 and 5.

### Example 3E

### Plasma Free Fatty Acid and Triglyceride Levels

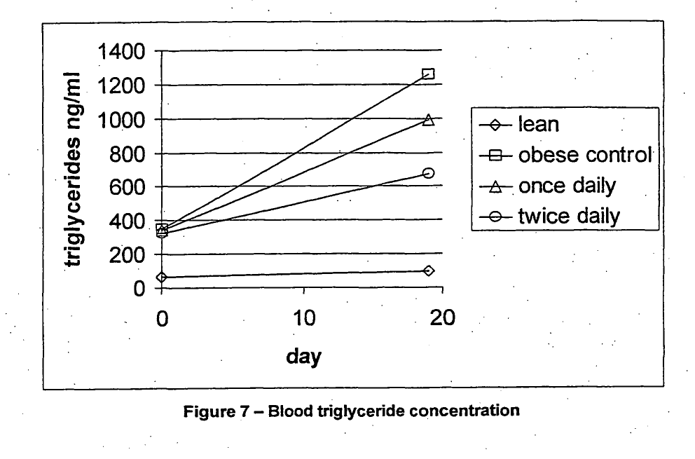

Plasma free fatty acid and triglyceride levels are significantly elevated in the obese animals at day 0, and in control obese animals they increase throughout the study period. Once-daily, and particularly twice-daily treatment attenuates this increase. The results are presented in FIGS. 6 and 7.

The results described above clearly indicate that long-term inhibition of DP-IV is effective in delaying the onset of diabetic symptoms in the ZDF rat, and hence that inhibitors of DP-IV should be useful as prophylactic agents for people at risk of developing type 2 diabetes and as a treatment for people in the early stages of the disease to delay the progression of diabetic complications.

1 The use, in the preparation of a pharmaceutical composition for treating an individual at risk of developing type 2 diabetes, or in the early stages thereof, so as to delay the onset and progression of the disease, of an inhibitor of dipeptidyl peptidase IV or a prodrug thereof, in which the inhibitor of dipeptidyl peptidase IV or prodrug thereof is a compound according to general formula 1 or a pharmaceutically acceptable salt thereof 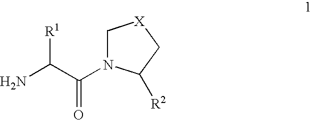
wherein: X is selected from CH₂ and S;
R¹ is selected from C₁-C₆ alkyl and (CH₂)ₙ)R³;
R² is CN;
R³ is selected from NH-Het and NHCO-Het; and
Het is a pyridyl, pyrimidyl or pyrazinyl group that is optionally substituted with up to two groups independently selected from methyl, CL, F, CN and CF₃; and
n is 2, 3, 4 or 5.

2 The use according to claim 2 in which X is CH₂.

3 The use according to claim 1 or 2 in which the inhibitor of dipeptidyl peptidase IV or prodrug thereof is an α-aminoacyl pyrrolidinenitrile.

4 The use according to claim 1, 2 or 3 in which the inhibitor of dipeptidyl peptidase IV or prodrug thereof is (2S)-1-((2′S)-2′-amino-3′,3′-dimethylbutanoyl)pyrrolidine-2-carbontrile or pharmaceutically active salt thereof.

5 The use according to claim 1, 2 or 3 in which the inhibitor of dipeptidyl peptidase IV or prodrug thereof is (2S)-1-((2′S)-2′-amino-5′-pyrazinecarbonylaminopentanoyl)pyrrolidine-2-carbonitrile or pharmaceutically active salt thereof.

6 The use according to any of claims 1 to 5 in which the pharmaceutical composition releases the inhibitor of dipeptidyl peptidase IV or prodrug thereof over a period of between one week and three months.

7 The use according to claim 6 in which the pharmaceutical composition is a depot formulation.

8 A method of treating an individual at risk of developing type 2 diabetes, or in the early stages thereof, so as to delay the onset and progression of the disease, which comprises the administration to the individual of an inhibitor of dipeptidyl peptidase IV or a prodrug thereof in which the inhibitor of dipeptidyl peptidase IV is a compound according to general formula 1 or a pharmaceutically acceptable salt thereof 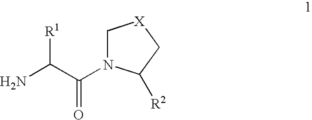
wherein: X is selected from CH₂ and S;
R¹ is selected from C₁-C₆ alkyl and (CH₂)ₙ)R³;
R² is CN;
R³ is selected from NH-Het and NHCO-Het; and
Het is a pyridyl, pyrimidyl or pyrazinyl group that is optionally substituted with up to two groups independently selected from methyl, Cl, F, CN and CF₃; and
n is 2, 3, 4 or 5.

9 A method according to claim 8 in which repeated doses of the inhibitor of dipeptidyl peptidase IV or prodrug thereof are administered.

10 The use according to any of claims 1 to 7, or a method according to any of claims 8 or 9, in which the individual is a human.

11 The use according to any of claims 1 to 7 or 10, or a method according to any of claims 8 to 10, to delay the onset of the disease.
